(19) United States
(12) Patent Application Publication
(10) Pub. No.: US 2019/0099122 A1
(43) Pub. Date: Apr. 4, 2019

(54) DETECTION OF ALLERGEN EXPOSURE

(71) Applicant: HEWLETT PACKARD ENTERPRISE DEVELOPMENT LP, Houston, TX (US)

(72) Inventors: Satwant KAUR, Mountain View, CA (US); Larry SCHMIDT, Atlanta, GA (US)

(73) Assignee: ENT. SERVICES DEVELOPMENT CORPORATION LP, Tysons, VA (US)

(21) Appl. No.: 15/539,620

(22) PCT Filed: Dec. 23, 2014
(86) PCT No.: PCT/US2014/072055

§ 371 Date: Jun. 23, 2017

(51) Int. Cl. A61B 5/00; G06Q 50/22; A61B 5/1459; A61B 5/157; G01N 33/52; A61B 5/15; A61B 5/155; G01N 33/542; G01N 33/543

(52) U.S. Cl. CPC A61B 5/411; G06Q 50/22; A61B 5/1459; A61B 5/157; G01N 33/52; G01N 2800/24; A61B 5/155; A61B 5/4839; G01N 33/542; G01N 33/54326; A61B 5/150022

(57) ABSTRACT

One example implementation includes an extractor controller to control extraction of blood from a user; a detection fluid manager to mix the blood and a detection fluid to form magnetized test particles, the detection fluid comprising dye coated magnetic particles; a particle flow controller to remove magnetized test particles from the mixed blood and detection fluid; and an allergen detector to detect exposure to an allergen based on a color of the test particles.

### BACKGROUND

There are various types of allergies, such as food (e.g., peanuts, milk, seafood, etc.), drug (e.g., penicillin), or seasonal allergies (e.g., to grass, weed, pollen, molds, etc.). Depending on the severity of an individual's sensitivity to allergens, allergic reactions may have various affects, from wheezing, swelling, itching, etc. and in the most severe cases the affects may be fatal. Allergic reactions may begin occurring in an individual upon contact or proximity to a particular allergen. Allergens may cause a presence or increase of histamines in a user's blood stream or body.

### BRIEF DESCRIPTION OF THE DRAWINGS

FIG. 1 is a block diagram of an example allergy detection system, including an example blood extractor, an example blood analyzer, and an example reaction preventer, constructed in accordance with the teachings of this disclosure.

FIG. 2 is a block diagram of an example blood analyzer that may be implemented by the allergy detection system in accordance with an aspect of this disclosure.

FIG. 3 is a block diagram of a reaction preventer that may be implemented by the allergy detection system in accordance with an aspect of this disclosure.

FIG. 4 is diagram of an example device that may be used to implement the allergy detection system of FIG. 1 in accordance with an aspect of this disclosure.

FIG. 5 is a flowchart representative of example machine readable instructions that may be executed to implement the allergen detection system of FIG. 1 or 4 in accordance with an aspect of this disclosure.

FIG. 6 is a flowchart representative of example machine readable instructions that may be executed to implement the blood analyzer of FIG. 1 or 2 in accordance with an aspect of this disclosure.

FIG. 7 is a flowchart representative of example machine readable instructions that may be executed to implement the reaction preventer of FIG. 1 or 3 in accordance with an aspect of this disclosure.

FIG. 8 is a block diagram of a processor platform capable of executing the instructions of FIGS. F1 or F2 to implement the allergy detection system of FIG. 1, 2, 3, or 4 in accordance with an aspect of the disclosure.

### DETAILED DESCRIPTION

Examples disclosed herein involve allergy detection using a device (e.g., a wearable device such as a ring, bracelet, or watch) to monitor blood of a user. The blood of the user may be mixed with a detection fluid including dye coated magnetic particles to form test particles that when encountered with allergens change color or intensity of light reflections. Accordingly, the example test particles, when analyzed with a photodetector in accordance with the teachings of the disclosure, may indicate the presence of allergens in the user's blood or whether the user is experiencing (or about to experience) an allergic reaction. In some examples, when allergens are detected in a user's blood, notifications may be sent to the user (or other individuals) indicating the exposure to the allergens or treatment may be provided to limit effects of the exposure to the allergens (e.g., to limit or prevent an allergic reaction).

Many individuals have severe allergies to foods, animals, plants, etc. In some examples, an individual's allergies may be so severe that allergic reactions may cause death. For example, if certain individuals with peanut allergies are in the same environment (e.g., room, building, etc.) as peanuts, the individuals run a risk of an extremely severe allergic reaction that may be fatal. In many of these cases, such a severe reaction may have been prevented had the user known that he or she was in the presence of an allergen sooner than when the user started experiencing symptoms of the allergic reaction. Examples disclosed herein provide for detection of allergens in the user's blood before the user may even realize that he or she is in the presence of an allergen. Example methods, apparatus, and articles of manufacture involve continuously monitoring a user for a period of time for the presence of an allergen within the user's blood. Examples disclosed herein may be included on a wearable device providing minimal invasiveness to maintain comfort but ensure safety of the individual by monitoring their blood for allergens.

An example system includes an extractor controller to control extraction of blood from a user; a detection fluid manager to mix a solution comprising the blood and magnetic particles; a particle flow controller to attract magnetic particles from the solution, the extracted magnetic particles comprising a dye coating; and a characteristic analyzer to detect an allergic reaction based on the dye coating.

As used herein, a user is exposed to an allergen (i.e., a protein substance that triggers an allergic reaction in an individual sensitized to the allergen) when a level of histamine is detected in a user's blood. A user may be exposed to an allergen by coming in contact with an allergen directly or by being within a proximity of the allergen (e.g., within a certain distance, with a same room, within a same building, etc.). As used herein, a user may experience an allergic reaction (e.g., swelling, congestion, wheezing, etc.) when the user is exposed to an allergen for a period of time, which may vary depending on the user's sensitivities to the allergen.

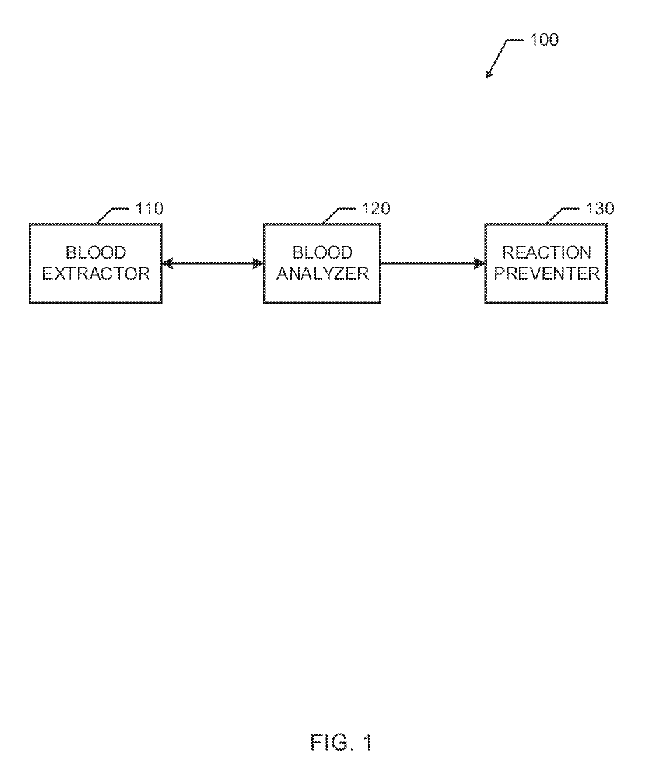

FIG. 1 is a block diagram of an example allergy detection system 100, including an example blood extractor 110, an example blood analyzer 120, and an example reaction preventer 130. In the illustrated example of FIG. 1, the blood extractor 110 communicates with the blood analyzer 120, the blood analyzer 120 communicates with the blood extractor 110 and the reaction preventer 130, and the reaction preventer 130 communicates with the blood analyzer 120. The example allergy detection system 100 of FIG. 1 may be included in a single device (e.g., within a housing of a single device) or within a plurality of devices. For example, a first component of the system 100, such as the blood extractor 110 or the blood analyzer 120, may include components located on a first device (e.g., a wearable device, such as a smart bracelet or smart watch), and a second component, such as the blood analyzer 120 or the reaction preventer 130, may include components on another device (e.g., a mobile device, such as a smartphone)). In examples disclosed herein, the allergy detection system 100 of FIG. 1 may be implemented by or configured on a wearable device of a user. Accordingly, the example allergy detection system 100 may continuously operate to detect allergic reactions of the user while the user is wearing a wearable device including the allergy detection system 100. In some examples, the allergy detection system 100 may be powered by batteries or by devices that capture or store power from a user's expended energy (e.g., movement, heat, etc.).

The example blood extractor 110 of FIG. 1 extracts blood from a user. In examples disclosed herein, the blood extractor 110 may include an extraction cannula that may be inserted into a body of a user (e.g., subcutaneously). The example extraction cannula may be configured within the allergy detection system 100 such that the extraction cannula extracts blood from a user and provides the blood to the blood analyzer 120. The example extraction cannula of the blood extractor 110 may be controlled to extract blood using any suitable technique. For example, actuators of the blood extractor 110 may be activated to extract the blood from the user via the extraction cannula. The example blood extractor 110 may extract blood in response to instructions from the blood analyzer 120. In some examples, the extraction cannula may be inserted into a user's body or skin using any suitable means, such as a spring loaded mechanism, an actuator, etc. In examples disclosed herein, the extraction cannula may remain subcutaneous while a user is wearing a device (e.g., a bracelet, a ring, a watch, etc.) including the allergy detection system 100.

The blood extractor 110 of FIG. 1 may include an extraction pad. For example, the extraction pad may be implemented by a replaceable cartridge of the allergy detection system 100 that a user may periodically or aperiodically replace.

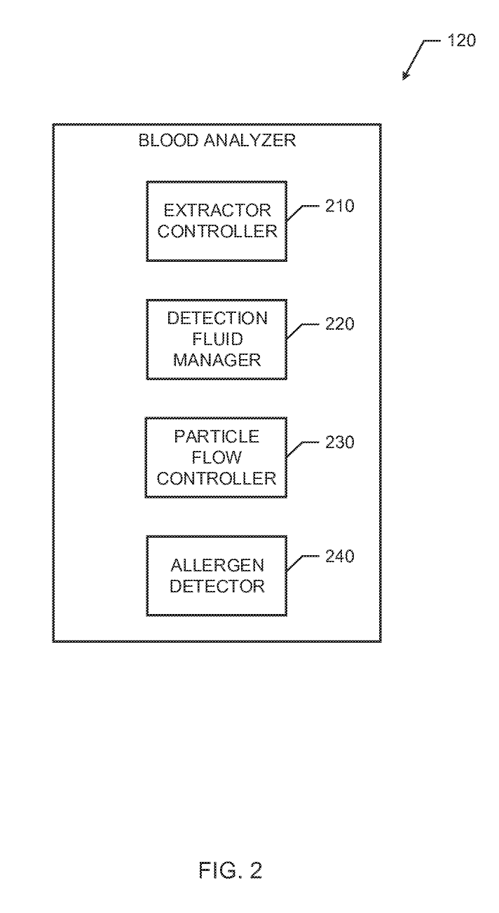

The example blood analyzer 120 of FIG. 1 analyzes blood extracted from the user by the blood extractor 110. The blood analyzer of FIG, 1 may instruct or control the blood extractor 110 to extract the blood periodically or aperiodically, depending on settings of the allergy detection system 100. The example blood analyzer 120 analyzes blood from the user in accordance with the teachings of this disclosure. An example implementation of the blood analyzer 120 is discussed herein in connection with FIG. 2.

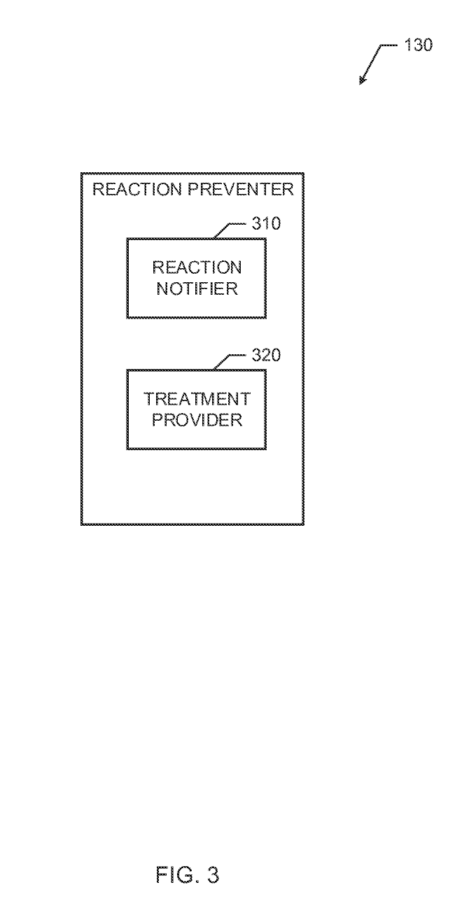

The example reaction preventer 130 of FIG. 1 takes action to prevent or limit an allergic reaction of a user. In some examples, the reaction preventer 130 may notify a user or another person (e.g., a relative, a healthcare provider, etc.) of exposure to an allergen or a potential allergic reaction. Additionally or alternatively, the allergic reaction preventer 130 may administer reaction prevention drug(s) (e.g., anti-histamine) to a user (e.g. via a treatment cannula). An example implementation of the reaction preventer 130 is discussed herein in connection with FIG. 3.

While an example manner of implementing the allergy detection system 100 is illustrated in FIG. 1, at least one of the elements, processes or devices illustrated in FIG. 1 may be combined, divided, re-arranged, omitted, eliminated or implemented in any other way. Further, the blood extractor 110, the blood analyzer 120, the reaction preventer 130, or more generally, the allergy detection system 100 of FIG. 1 may be implemented by hardware or any combination of hardware and executable instructions (e.g., software or firmware). Thus, for example, any of the blood extractor 110, the blood analyzer 120, the reaction preventer 130, or more generally, the allergy detection system 100 of FIG. 1 could be implemented by or include at least one of an analog or digital circuit, a logic circuit, a programmable processor, an application specific integrated circuit (ASIC), a programmable logic device (PLD) or a field programmable logic device (FPLD). When reading any of the apparatus or system claims of this patent to cover a purely software or firmware implementation, at least one of the blood extractor 110, the blood analyzer 120, or the reaction preventer 130 is/are hereby expressly defined to include a tangible computer readable storage device or storage disk such as a memory, a digital versatile disk (DVD), a compact disk (CD), a Blu-ray disk, etc. storing the executable instructions. Further still, the example allergy detection system 100 of FIG. 1 may include at least one element, process, or device in addition to, or instead of, those illustrated in FIG. 1, or may include more than one of any or all of the illustrated elements, processes, and devices.

FIG. 2 is a block diagram of an example blood analyzer 120 that may be implemented by the allergy detection system 100. The example blood analyzer 120 of FIG. 2 may be used to implement the blood analyzer 120 of FIG. 1. The example blood analyzer 120 of FIG. 2 includes an extractor controller 210, a detection fluid manager 220, a particle flow controller 230, and an allergen detector 240. In examples disclosed herein, the blood analyzer 120 of FIG. 2 analyzes blood extracted from a user to detect whether the user has been exposed to an allergen or is experiencing an allergic reaction.

The example extractor controller 210 of FIG. 2 controls a blood extractor (e,g., the blood extractor 110 of FIG. 1) to extract blood from a user. For example, the extractor controller 210 may instruct the blood extractor 110 to extract blood from a user via an extraction cannula. In some examples, the extractor controller 210 may use a timer to instruct the blood extractor 110 to extract the blood periodically (e.g., every 30 minutes, hourly, every 4 hours, etc.) Settings of the timer may be adjustable based on instructions from a user received via a user interface of the allergy detection system 100. In some examples, different periods of time may be used based on timing in the day or during the calendar year. For example, the extractor controller 210 may instruct the blood extractor 110 to extract blood more frequently during daytime hours when a user is eating versus nighttime hours when a user is not eating.

The example detection fluid manager 220 of FIG. 2 controls mixing of blood extracted by the blood extractor 110 with a detection fluid, such as a histamine detection fluid. The example detection fluid manager 220 may control a flow of detection fluid from a detection fluid container or a detection fluid pad. For example, the detection fluid manager 220 may control the flow of histamine detection fluid from the detection fluid container to a mixing container or mixing pad (e.g., a blood collection pad). An example histamine detection fluid may include magnetic particles coated with histamine antibodies and a dye (e.g., phenylenediamine) that changes color (e.g., an intensity of reflected light) when exposed to both histamines and histamine antibodies. The example dye coating of the histamine antibodies, in accordance with the teachings of this disclosure, allow the magnetic particles to attract histamines that may be present in blood extracted by the blood extractor 110. Accordingly, when the detection fluid is mixed with blood including histamines caused by exposure to allergens, any histamines in the blood may be bonded to the histamine antibodies, and, due to the dye (e.g., a histamine-sensitive dye), the color of the particles change. In other examples, other types of blood analysis other than a histamine analysis may be performed. As used herein, after the blood is mixed with the magnetic particles, the magnetic particles are referred to as test particles or magnetic test particles. The example test particles include magnetic particles and a coating indicative of the presence or absence of an allergic reaction (e.g., based on whether histamines reacted with histamine-antibodies of the detection fluid).

The example particle flow controller 230 controls the flow of test particles within the allergy detection system 100. The example test particles have a coating and the color of that coating depends on whether histamines were present in a user's blood. The particle flow controller 230 of FIG. 2 may include a magnet or an electromagnet. For example, the particle flow controller 230 may apply a current to an electromagnet to attract test particles toward the electromagnet. In some examples, the particle flow controller 230 may include a particle stopper (e.g., a valve, actuator, etc.) to stop particles in place (e.g., in a location where the particles may be analyzed). Accordingly, after mixing the blood with the detection fluid, the particle flow controller 230 may control the flow of the test particles from the detection fluid to move toward a location of the allergy detection system 100 for analysis (e.g., a location of the allergen detector 240).

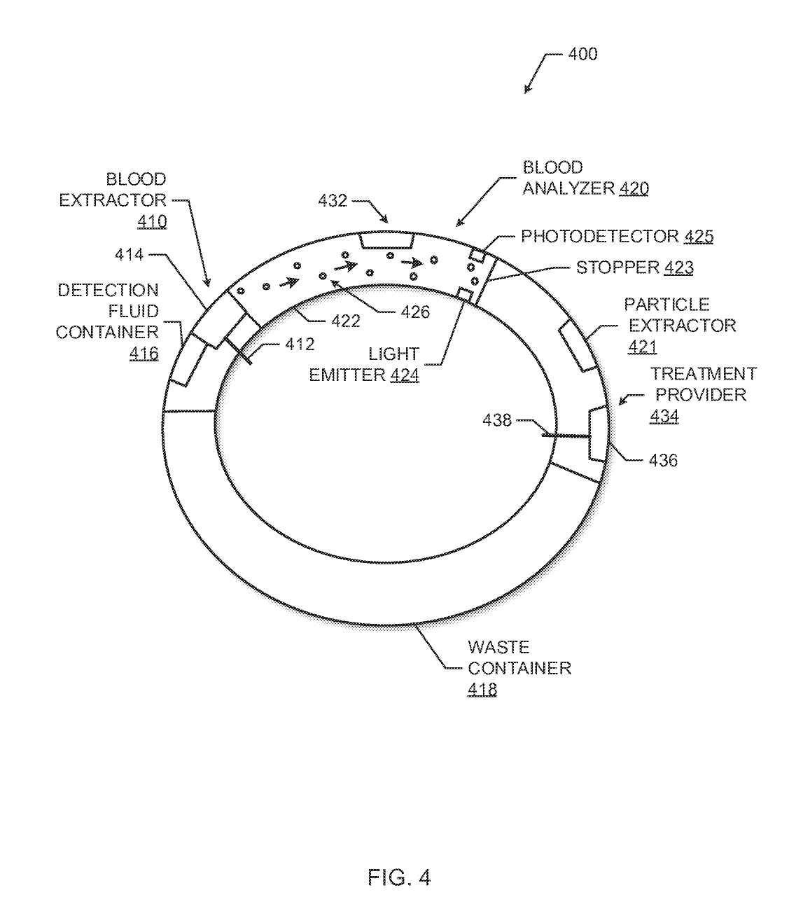

The example allergen detector 240 of FIG. 2 detects whether histamine is located in the blood extracted by the blood extractor 110 and mixed with the detection fluid. The example allergen detector 240 may include a light emitter and a photodetector for measuring or determining a color of the coating on the magnetic particles (see FIG. 4). The allergen detector 240 may then determine whether histamine is present in the extracted blood based on the color of the coating of the magnetic particles, and, therefore, whether the user has been exposed to an allergen.

In some examples, the allergen detector 240 may compare a determined color of the coated magnetic particles with previous measurements of colors of previously analyzed magnetic particles from blood that was previously extracted from the user. For example, a baseline color or baseline measurement color may be measured from the user's blood when it is known that the user has not been exposed to an allergen for a period of time or is not undergoing an allergic reaction. Accordingly, the allergen detector 240 may subsequently measure the coated magnetic particles and compare the measured color to a previously measured color or colors. If there is a threshold difference (which may vary from user to user or be the same for each user) between the measured colors of the test particles between the baseline and another measurement resulting in an example detected allergen exposure color, the allergen detector 240 may determine that histamine is present in the extracted blood. In some examples, this allergen detector 240 or blood analyzer 120 may alert or notify the reaction preventer 130 that the user has been exposed to an allergen or that an allergic reaction is present or ongoing within the user.

In some examples, after histamine is detected in a user's blood, the blood analyzer 120 may instruct the blood extractor 110 to extract blood from the user again regardless of the schedule for blood extraction to confirm the results of the analysis. If a measurement from the subsequent extraction of blood yields similar results compared to those that were previously measured, it can be assumed that an allergic reaction is ongoing. However, in some examples, if the measurement from the subsequent extraction of blood indicates that no allergen or histamine is detected in the user's blood (e.g., the measurements are similar to those of the baseline measurements), then it may be assumed that the previous measurement indicating allergen exposure or an allergic reaction was flawed, and that no allergen exposure has occurred or no allergic reaction is occurring. In such examples, a notification of a presumed false positive may be sent to a user via the reaction preventer 130. In some examples, a number of false measurements may be monitored to confirm that the allergy detection system is operating properly and may indicate when a threshold number of false positives have been measured, indicating potential errors or failure of the allergy detection system 100.

In some examples, the blood analyzer 120 of FIG. 1 or 2 may control waste of the allergy detection system 100. For example, the blood analyzer 120 may move test particles, blood, detection fluid, or detection fluid/blood mixtures into a waste container of the allergy detection system 100. Accordingly, after blood is extracted from a user or test particles are tested by the blood analyzer 120, the blood or test particles may be discarded into a waste container that may be periodically or aperiodically emptied, cleaned, or replaced.

While an example manner of implementing the blood analyzer 120 of FIG. 1 is illustrated in FIG. 2, at least one of the elements, processes or devices illustrated in FIG. 2 may be combined, divided, re-arranged, omitted, eliminated or implemented in any other way. Further, the extractor controller 210, the detection fluid manager 220, the particle flow controller 230, the allergen detector 240, or more generally, the blood analyzer 120 may be implemented by hardware or any combination of hardware and executable instructions (e.g., software or firmware). Thus, for example, any of the extractor controller 210, the detection fluid manager 220, the flow controller 230, the allergen detector 240, or, more generally, the example blood analyzer 120 could be implemented by at least one of an analog or digital circuit, a logic circuit, a programmable processor, an application specific integrated circuit (ASIC), a programmable logic device (PLD) or a field programmable logic device (FPLD). When reading any of the apparatus or system claims of this patent to cover a purely software or firmware implementation, at least one of the extractor controller 210, the detection fluid manager 220, the flow controller 230, or the allergen detector 240 is/are hereby expressly defined to include a tangible computer readable storage device or storage disk such as a memory, a digital versatile disk (DVD), a compact disk (CD), a Blu-ray disk, etc. storing the executable instructions. Further still, the example blood analyzer 120 of FIG. 2 may include at least one element, process, or device in addition to, or instead of, those illustrated in FIG. 2, or may include more than one of any or all of the illustrated elements, processes, and devices.

FIG. 3 is a block diagram of a reaction preventer 130 that may be implemented by the allergy detection system 100 of FIG. 1. The example reaction preventer 130 of FIG, 3 may be used to implement the reaction preventer 130 of FIG. 1. The example reaction preventer 130 includes a reaction notifier 310 and a treatment provider 320. In examples disclosed herein, when the reaction preventer 130 is notified of an occurring allergic reaction (e.g., by the blood analyzer 120), the reaction preventer 130 performs operations to prevent or minimize the effects of the allergic reaction.

The example reaction notifier 310 of FIG. 3 notifies a user or another party (e.g., a family member, a healthcare provider, or any other person or institution selected by the user) of an occurring allergic reaction. Accordingly, the reaction notifier 310 may include a user interface to facilitate user input (e.g., a keypad, buttons, a touchscreen) or user output (e.g., a light emitting diode (LED), a display screen, etc.). In some examples, the reaction notifier 310 may include a wireless transmitter or antenna to facilitate communication with another device. For example, the reaction notifier 310 may establish a communication link (e.g., a Bluetooth™ link, a Wi-Fi link, etc.) with a smartphone or other device. The example smartphone or device (e.g., via an application) may then be used to communicate with other parties selected by the user (e.g., relatives, healthcare providers, emergency care facilities, etc.), for example using the Internet or cellular phone system.

In some examples, a user interface of the reaction notifier 310 may be used to alert users in connection with the allergy detection system 100. For example, the reaction notifier 310 may alert the user (e.g., via an LED, a display, a message to a mobile device, etc.) that the allergy detection system 100 is in need of more detection fluid, a new extraction pad, emptying or replacing of a waste container, etc. Accordingly, the reaction notifier 310 may provide maintenance notifications of the allergy detection system to a user.

The example treatment provider 320 of FIG. 3 may control or provide treatment to the user upon detection of an allergic reaction by the blood analyzer 120. In examples disclosed herein, the treatment provider 320 may include a cannula and drug cartridge that includes drugs (e.g., anti-histamines) that may be administered or injected into a user to prevent or limit effects of the detected allergic reaction. For example, the allergy detection system 100 may include a drug storage cartridge mechanically connected to a drug cannula that may be controlled by the treatment provider 320 to inject drug from the cartridge into the user via the cannula in response to detection of an allergic reaction by the blood analyzer 120. In examples disclosed herein, the cannula may be inserted into the user using any suitable techniques and may remain subcutaneously inserted into the user while the user wears a device including the allergy detection system 100.

In some examples, a dosage of a drug injected into the user by the treatment provider 320 may be determine based on a severity of the detected allergic reaction. In such examples, the blood analyzer 120 may provide an estimated amount of histamine or level of severity of the allergic reaction based on the detected color of the test particles or color change between a set of analyzed test particles. Accordingly, the treatment provider 320 of the allergy detection system 100 allows for near immediate treatment of an allergic reaction and as needed treatment of an allergic reaction depending on the severity of the allergic reaction.

While an example manner of implementing the treatment provider 320 of FIG. 1 is illustrated in FIG. 3, at least one of the elements, processes or devices illustrated in FIG. 3 may be combined, divided, re-arranged, omitted, eliminated or implemented in any other way. Further, the reaction notifier 310 or the treatment provider 320 or, more generally, the example reaction preventer 130 of FIG. 3 may be implemented by hardware or any combination of hardware and executable instructions (e.g., software or firmware). Thus, for example, any of reaction notifier 310 or the treatment provider 320, or, more generally, the example reaction preventer 130 could be implemented by at least one of an analog or digital circuit, a logic circuit, a programmable processor, an application specific integrated circuit (ASIC), a programmable logic device (PLD) or a field programmable logic device (FPLD). When reading any of the apparatus or system claims of this patent to cover a purely software or firmware implementation, at least one of reaction notifier 310 or treatment provider 320 is/are hereby expressly defined to include a tangible computer readable storage device or storage disk such as a memory, a digital versatile disk (DVD), a compact disk (CD), a Blu-ray disk, etc. storing the executable instructions. Further still, the example reaction preventer 130 of FIG. 3may include at least one element, process, or device in addition to, or instead of, those illustrated in FIG. 3, or may include more than one of any or all of the illustrated elements, processes and devices.

FIG. 4 is a diagram of an example allergy detection device 400 constructed in accordance with the teachings of this disclosure. The example allergy detection device 400 may be used to implement the allergy detection system 100 of FIG. 1. The example allergy detection device 400 includes a blood extractor 410 (which may be used to implement the blood extractor 110 of FIG. 1), a blood analyzer 420 (which may be used to implement the blood analyzer 120 of FIG. 1), a reaction notifier 432 and a treatment provider 434 (both of which may be used to implement the reaction preventer 130 of FIG. 1). The example components 410, 420, 432, or 434 of FIG. 4 may include control hardware or a combination of control hardware and computer readable instructions that when executed may detect or treat allergic reactions in accordance with the teachings of this disclosure.

In the illustrated example of FIG. 4, the blood extractor 410 includes an extraction cannula 412 and an extraction pad 414. The extraction cannula 412 may be subcutaneously inserted into a body part (e.g., a finger, a wrist, etc.) of a user using any suitable means (e.g., actuator(s), needle(s), etc.). The example extraction pad 414 may store or absorb blood extracted by the extraction cannula 412. The example blood extractor 410 may be controlled (e.g., by an extractor controller of the blood analyzer 420, such as the extraction controller 210 of FIG. 2) to periodically or aperiodically extract blood from the user via the extraction cannula 412 to be stored (e.g., temporarily) by the extraction pad 414. In some examples, the extraction pad 414 may include or be comprised of a replaceable cartridge that may be removed after a period of time (e.g., one month) or a number of extractions of blood (e.g., 100, 1000, etc.). The extraction pad 414 may also receive detection fluid from a detection fluid container 416. The example detection fluid container 416 may release detection fluid periodically or aperiodically using a valve or actuator between the detection fluid container 416 and the extraction pad 414 (e.g., after each extraction of blood from a user via the extraction cannula 412). The example valve or actuator of the detection fluid from the detection fluid container 416 may be controlled by the blood analyzer 420 (e.g., via a detection fluid manager, such as the detection fluid manager 220). In the illustrated example of FIG. 4, waste (e.g., excess blood or detection fluid) from the extraction pad 414 or detection fluid container 416 may be released (e.g., via a valve or actuator) into a waste container 418.

The example waste container 418 of FIG. 4 may be a replaceable cartridge or detachable cartridge that may be replaced with new cartridges or cleaned, respectively, as needed. For example, when the waste container 418 is filled with blood, detection fluid, or test particles, a user may replace or clean the waste container 418 to allow for continued operation of the allergy detection device 400 of FIG. 4. In some examples, when the waste container 418 is at capacity with waste (e.g., blood, detection fluid, or test particles), the allergy detection device 400 may cease extracting blood or analyzing test particles until the waste container 418 is replaced or cleaned. In such examples, a blood analyzer 120 (e.g., the blood analyzer 420) or reaction preventer 130 (e.g., the reaction notifier 432) of the allergy detection device may monitor a level of waste in the waste container 432 (e.g., using pressure sensors, fluid sensors, etc.).

The example blood analyzer 420 of FIG. 4 includes a particle extractor 421, a flow chamber 422, a stopper 423, and a light emitter 424, and a photodetector 425. In the illustrated example of FIG. 4, the particle extractor 421, flow chamber 422, and stopper 423 may be controlled (e.g., using valves, actuators, electrical signals, etc.) by a particle flow controller, such as the particle flow controller 230 of FIG. 2. As illustrated by the arrows in FIG. 4, test particles 426 flow towards the particle extractor 421 through the flow chamber 422 until they are stopped by the stopper 423. The example particle extractor may be an electromagnet that receives a signal from the blood analyzer 420 to activate the magnet to attract the magnetic test particles from a mixture of detection fluid and blood in the extraction pad 414. Accordingly, the blood analyzer 420 may allow the flow of test particles from the extraction pad 414 through the flow chamber 422 until the test particles reach the stopper 423. The extraction pad 414 may be located within the flow chamber 422 or a valve or actuator between the extraction pad and the flow chamber 422 may be opened or activated to allow the flow of the test particles 426 through the flow chamber 422.

In the illustrated example of FIG. 4, the blood analyzer 420 may stop the flow of the test particles using the stopper 423 near the light emitter 424 and photodetector 425 for analysis of the test particles. The example stopper 423 may be a valve or actuator. In examples disclosed herein, the light emitter 424 may emit bursts of light toward the test particles such that the photodetector 425 may determine a color of the test particles (i.e., a color of a coating of the test particles). The example photodetector 425 may determine a color of the test particles based on a measurement of voltage from the light emitted by the light emitter toward the test particles. A color of the test particles affects the voltage measurement, and thus any changes in color may be detected from one measurement of the test particles to the next. In examples disclosed herein, the blood analyzer 420 may compare measurements to determine a change in characteristics of the blood. For example a first measurement may have been taken (e.g., a baseline measurement) when it is known that there are not histamines in a user's blood and a second measurement may be later taken that results in a different color of test particles than was determine in the first measurement. Accordingly, based on the change in color detected between the first measurement and the second measurement, the blood analyzer 420 may determine that histamines are present in the user's blood from the second measurement and therefore exposure to an allergen or an allergic reaction has occurred or is occurring. After analyzing the test particles 426, the blood analyzer 420 may release the test particles 426 into the waste container 418.

The example reaction notifier 432 and the treatment provider 434 of FIG. 4 may be used to implement the reaction preventer 130 of FIG. 1. In the illustrated example of FIG. 4, the reaction notifier 432 is located on an outside of the allergy detection device 400 and may include LEDs or a display device to send alerts in connection with the allergy detection device 400. For example, the alerts may indicate the presence of an allergic reaction in the user, maintenance needed for the allergy detection device 400, etc. In some examples, the reaction notifier 432 includes a transceiver for communicating with another device such as a mobile device (e.g. smartphone, table computer, personal digital assistant, etc.).

The example treatment provider 434 of FIG. 4 includes a drug container 436 and treatment cannula 438. The example drug container 436 may store a drug (e.g., an antihistamine) that may be injected into a user via the cannula 438 upon detection of an allergen by the blood analyzer 420 in accordance with the teachings of this disclosure. In some examples, the cannula may be inserted subcutaneously in the user and remain inserted in the user while the user is wearing the allergy detection device 400. In some examples, the cannula may be inserted into the skin of the user in response to detecting the allergen or histamine in the user's blood. The example treatment provider 434 may also include a monitor to inject the drug from the drug container 434 in doses corresponding to a severity of the allergic reaction. For example, the more severe a detected allergic reaction is the greater an amount of the drug that is injected into the user.

Accordingly, the allergy detection device 400, which may be a wearable device (e.g., a ring, a bracelet, a watch, etc.), may be used to implement the allergy detection system 100 of FIG. 1. The allergy detection device 400 includes exemplary components to facilitate extraction of blood, movement of coated magnetic test particles, analysis of the test particles, allergic reaction detection and alert capabilities, allergic reaction treatment, and waste management of byproducts of the allergy detection system 100 of FIG. 1 in accordance with the teachings of this disclosure. Other example devices, components, or layouts of the example components of the allergy detection device 400 may be used to achieve the purposes of this disclosure.

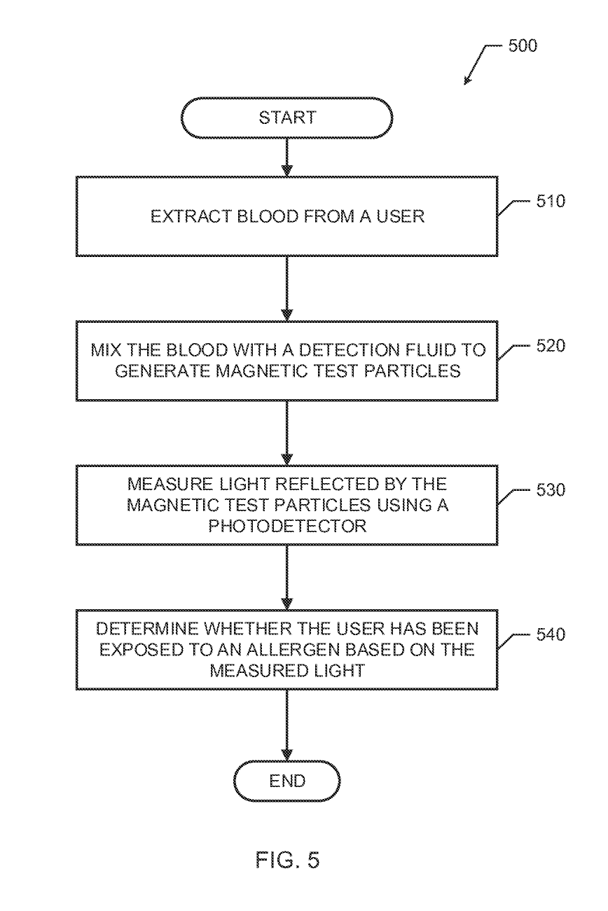

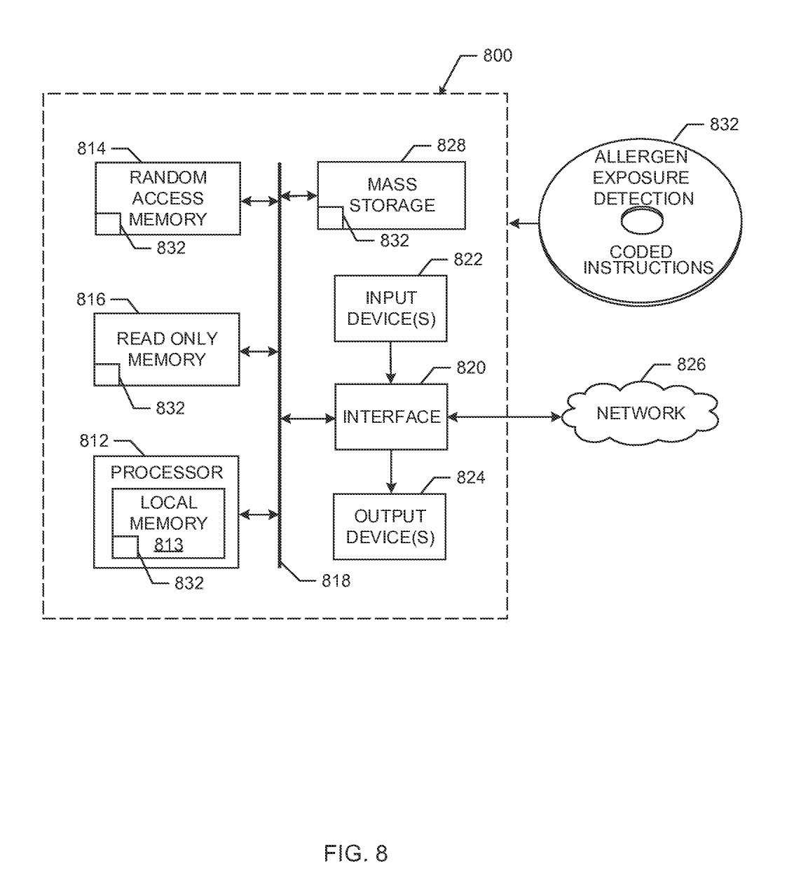

Flowcharts representative of example machine readable instructions for implementing the allergy detection system 100 of FIG. 1 are shown in FIGS. 5, 6, and 7. The machine readable instructions comprise program(s)/process(es) for execution by a processor such as the processor 812 shown in the example processor platform 800 discussed below in connection with FIG. 8. The program(s)/process(es) may be embodied in executable instructions (e.g., software) stored on a tangible computer readable storage medium such as a CD-ROM, a floppy disk, a hard drive, a digital versatile disk (DVD), a Blu-ray disk, or a memory associated with the processor 812, but the entirety of the program(s)/process(es) or parts thereof could alternatively be executed by a device other than the processor 812 or embodied in firmware or dedicated hardware. Further, although the example program(s) is/are described with reference to the flowcharts illustrated in FIG. 5, 6, or 7, many other methods of implementing the example allergy detection system may alternatively be used. For example, the order of execution of the blocks may be changed, or some of the blocks described may be changed, eliminated, or combined,

The process 500 of FIG. 5 begins with an initiation of the allergy detection system 100 (e.g., upon startup, upon instructions from a user, upon startup of a device implementing the allergy detection system 100 (e.g., the allergy detection device 400), etc.). At block 510 of FIG. 5, the blood extractor 110 extracts blood from a user. At block 520, the blood analyzer 120 mixes the extracted blood with a detection fluid to generate magnetic test particles. The example detection fluid in block 520 may include magnetic particles coated with a dye that may change color based on characteristics of the blood (e.g., the presence of histamines or antihistamines in the blood). At block 530, the blood analyzer 120 measures light reflected by the magnetic test particles using a photodetector. At block 540, the blood analyzer 120 determines whether the user has been exposed to an allergen based on the measured light. In some examples, the blood analyzer 120 may make such a determination by comparing the measured to light to a previous measurement (e.g., a baseline measurement) of light from previously extracted blood or test particles. In some examples, the measurement of the light may be cross checked with a database of light measurements indicating whether or not the presence of an allergen corresponds to particular light measurements. The example database may be located with or managed by the allergy detection system 100 of FIG. 1. After block 540, the process 500 of FIG. 5 ends. In some examples, after block 540 of FIG. 5, the reaction preventer 130 may alert a user or provide treatment to the user when it is determined that the user has been exposed to an allergen or is experiencing an allergic reaction.

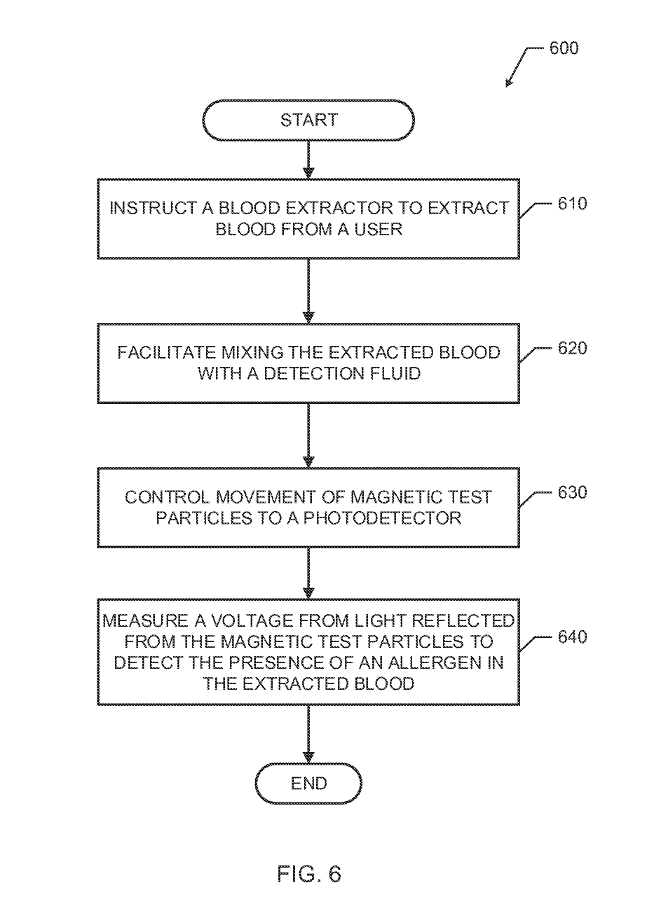

The process 600 of FIG. 6 begins with an initiation of the blood analyzer 120 (e.g., upon startup, upon instructions from a user, upon startup of a device implementing the blood analyzer 120 (e.g., the allergy detection system 100), etc.). At block 610, the extractor controller 210 instructs the blood extractor to extract blood from a user. At block 620, the detection fluid manager 220 facilitates mixing the extracted blood with a detection fluid. For examples, at block 620, the detection fluid manager 220 may control a valve or actuator of an allergy detection system 100 to release detection fluid onto an extraction pad comprising blood extracted from the user by the blood extractor 110. At block 630 of FIG. 6, the particle flow controller 330 controls movement of magnetic test particles formed from the mixture of the blood and detection fluid to a photo detector of the blood analyzer. At block 640, the allergen detector 240, via the photodetector, measures a voltage from light reflected from the magnetic test particles to detect the presence of an allergen in the extracted blood. The example allergen detector 240, at block 640, may compare the measured voltage to voltage measurements of previously test magnetic particles of previously extracted blood from the user or to a table of voltage measurements indicating whether or not the voltage measurement is indicative of an allergic reaction how severe of an allergic reaction the voltage measurement corresponds to. In examples disclosed herein, a severity of an allergic reaction may be based on a size difference in voltage measurement between previously measured voltages (e.g., from a baseline measurement) and a current measurement. In other words, the greater the voltage measurement (e.g., indicative of a greater color change of the test particles), the greater the severity of the allergic reaction.

The process 700 of FIG, 7 begins with an initiation of the reaction preventer 130 (e.g., upon startup, upon instructions from a user, upon startup of a device implementing the reaction preventer 130 (e.g., the allergy detection system 100), etc.). At block 710, the reaction preventer 130 determines whether an allergic reaction has been detected. For example, at block 710, the reaction preventer monitors for communications or notifications from the blood analyzer 120 indicating that the user is experiencing an allergic reaction or has been exposed to allergens. If the no allergic reaction has been detect, then control returns to block 710 to continue monitoring for allergic reaction detections. If at block 710, the reaction preventer 130 determines that an allergic reaction has been detected (e.g., in response to a notification from the blood analyzer 120), the reaction notifier 310 sends an alert to the user (block 720). In some examples, at block 720, the reaction notifier 310 may activate LEDs of the allergy detection system 100 or send a notification to another device (e.g., a mobile device) via a communication link (e,g., such as a Bluetooth™, Wi-Fi, etc.).

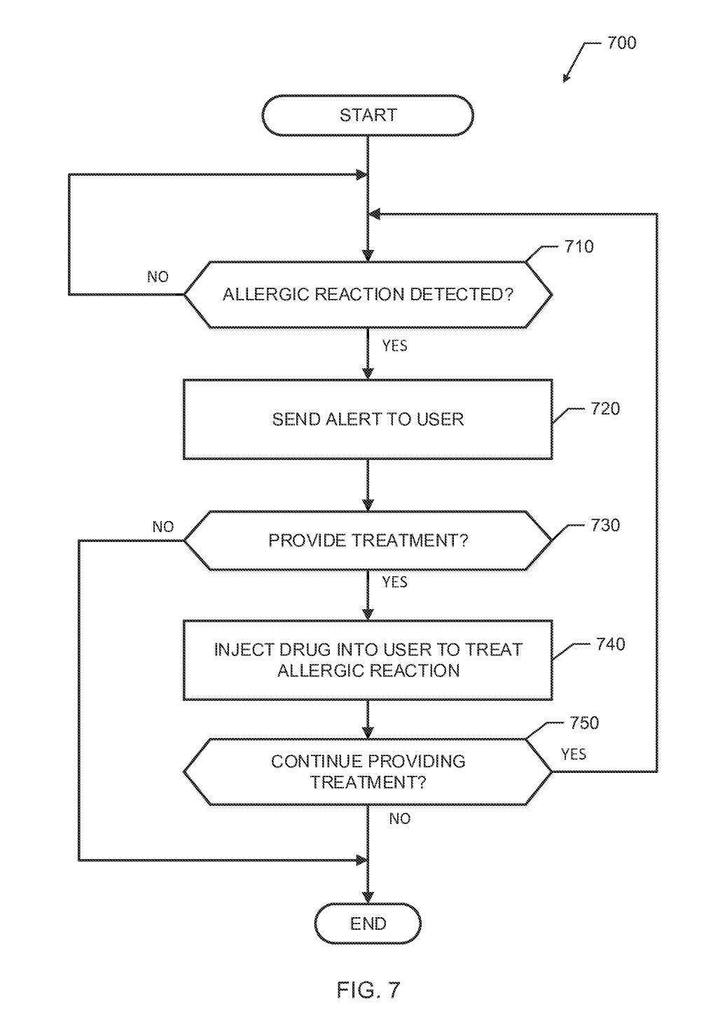

After block 720 in the illustrated example of FIG. 7, at block 730, the reaction preventer 130 determines whether to provide treatment to the user. For example, the reaction preventer 130 may check settings or capabilities of the allergy detection system 100 to determine whether to treat the detected allergic reaction. If no treatment is to be provided (e.g., the user placed settings indicating not to provide treatment, such treatment options are not operational (e.g., there is not sufficient resources or drugs to provide to the user), then the example process 700 of FIG. 7 ends. If, at block 730, the reaction preventer 130 determines that treatment is to be provided (e.g., there is enough drug to apply to the user), then at block 740, the treatment provider 320 injects a drug into the user to treat the allergic reaction. In some examples, at block 740, a dosage or amount of the drug injected in the user may depend on a severity of the allergic reaction or exposure to the allergen.

At block 750 of the example process 700 of FIG. 7, the treatment provider 320 determines whether to continue to provide treatment to the user. If the treatment provider 320 is to continue to provide treatment to the user, control returns to block 710. If the treatment provider 320 is no longer to continue to provide treatment to the user, the process 700 of FIG. 7 ends. For example, at block 750, the process 700 may end if the treatment provider 320 determines that the severity of the exposure to the allergen was low enough that the treatment provided in block 740 is sufficient enough to limit or stop the effects of an allergic reaction caused by the exposure to the allergen. Conversely, if the treatment provided at block 740 is not deemed to be enough to stop the allergic reaction, control may return to block 710 to receive additional measurement information from the blood analyzer 120.

As mentioned above, the example processes of FIG. 5, 6, or 7 may be implemented using coded instructions (e.g., computer or machine readable instructions) stored on a tangible computer readable storage medium such as a hard disk drive, a flash memory, a read-only memory (ROM), a compact disk (CD), a digital versatile disk (DVD), a cache, a random-access memory (RAM) or any other storage device or storage disk in which information is stored for any duration (e.g., for extended time periods, permanently, for brief instances, for temporarily buffering, or for caching of the information). As used herein, the term tangible computer readable storage medium is expressly defined to include any type of computer readable storage device or storage disk and to exclude propagating signals and to exclude transmission media. As used herein, “tangible computer readable storage medium” and “tangible machine readable storage medium” are used interchangeably. Additionally or alternatively, the example processes of FIG. 5, 6, or 7 may be implemented using coded instructions (e.g., computer or machine readable instructions) stored on a non-transitory computer or machine readable medium such as a hard disk drive, a flash memory, a read-only memory, a compact disk, a digital versatile disk, a cache, a random-access memory or any other storage device or storage disk in which information is stored for any duration (e.g., for extended time periods, permanently, for brief instances, for temporarily buffering, or for caching of the information). As used herein, the term non-transitory computer readable medium is expressly defined to include any type of computer readable storage device or storage disk and to exclude propagating signals and to exclude transmission media. As used herein, when the phrase “at least” is used as the transition term in a preamble of a claim, it is open-ended in the same manner as the term “comprising” is open ended. As used herein the term “a” or “an” may mean “at least one,” and therefore, “a” or “an” do not necessarily limit a particular element to a single element when used to describe the element. As used herein, when the term “or” is used in a series, it is not, unless otherwise indicated (e.g., when “or” is accompanied by the term “either”), considered an “exclusive or.”

FIG. 8 is a block diagram of an example processor platform 800 capable of executing the instructions of FIG. 5, 6, or 7 to implement the allergy detection system 100 or parts of the allergy detection system 100 of FIG. 1. The example processor platform 800 may be or may be included in any type of apparatus, such as a mobile device (e.g., a cell phone, a smart phone, a tablet, etc.), a personal digital assistant (PDA), an Internet appliance, a smart watch, a smart bracelet, or any other type of wearable device.

The processor platform 800 of the illustrated example of FIG. 8 includes a processor 812. The processor 812 of the illustrated example is hardware. For example, the processor 812 can be implemented by at least one integrated circuit, logic circuit, microprocessor or controller from any desired family or manufacturer.

The processor 812 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS Dynamic Random Access Memory (RDRAM) or any other type of random access memory device. The non-volatile memory 816 may be implemented by flash memory or any other desired type of memory device. Access to the main memory 814, 816 is controlled by a memory controller.

The processor platform 800 of the illustrated example also includes an interface circuit 820. The interface circuit 820 may be implemented by any type of interface standard, such as an Ethernet interface, a universal serial bus (USB), or a peripheral component interconnect (PCI) express interface.

In the illustrated example, at least one input device 822 is connected to the interface circuit 820. The input device(s) 822 permit(s) a user to enter data and commands into the processor 812. The input device(s) can be implemented by, for example, an audio sensor, a microphone, a camera (still or video), a keyboard, a button, a mouse, a touchscreen, isopoint or a voice recognition system,

At least one output device 824 is also connected to the interface circuit 820 of the illustrated example. The output device(s) 824 can be implemented, for example, by display devices (e.g., a light emitting diode (LED), an organic light emitting diode (OLED), a touchscreen), a tactile output device, speakers, etc. The interface circuit 820 of the illustrated example, thus, may include a graphics driver card, a graphics driver chip or a graphics driver processor.

The interface circuit 820 of the illustrated example also includes a communication device such as a transmitter, a receiver, a transceiver, a modem or network interface card to facilitate exchange of data with external machines (e.g., computing devices of any kind) via a network 826 (e.g., an Ethernet connection, a digital subscriber line (DSL), a telephone line, coaxial cable, a cellular telephone system, etc.).

The processor platform 800 of the illustrated example also includes at least one mass storage device 828 for storing executable instructions (e.g., software) or data. Examples of such mass storage device(s) 828 include floppy disk drives, hard drive disks, compact disk drives, Blu-ray disk drives, RAID systems, and digital versatile disk (DVD) drives.

The coded instructions 832 of FIG. 5, 6, or 7 may be stored in the mass storage device 828, in the local memory 813 in the volatile memory 814, in the non-volatile memory 816, or on a removable tangible computer readable storage medium such as a CD or DVD.

From the foregoing, it gill be appreciated that the above disclosed methods, apparatus and articles of manufacture involve monitoring for allergic reactions and treating detected allergic reactions using a wearable device. Accordingly, a wearable device, constructed in accordance with the teachings of this disclosure may continuously monitor a user's blood to detect allergic reactions, even before the user realizes that he or she is in the presence of any allergens. An example detection solution including coated magnetic particles may be mixed with the user's blood to form test particles that are analyzed using a photodetector to identify a color or color change between measurements of the test particles. Accordingly, response time, treatment time, and/or allergic reaction prevention may be achieved using the examples disclosed herein.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus comprising:
an extractor controller to control extraction of blood from a user;
a detection fluid manager to mix the blood and a detection fluid to form test particles, the detection fluid comprising dye coated magnetic particles;
a particle flow controller to remove test particles from the mixed blood and detection fluid; and
an allergen detector to detect exposure to an allergen based on a color of the test particles, the color resulting from the mixture of the blood and dye coated magnetic particles.

2. The apparatus of claim 1, wherein the dye coated magnetic particles comprise a dye coating including histamine antibodies that change color when exposed to histamines and antibodies of the histamines, and
the allergen detector detects the exposure to the allergen based on a change in color of the test particles from a baseline measurement color to an allergen exposure color due to the presence of histamines in the blood.

3. The apparatus of claim 2, wherein the allergen detector comprises:
a light emitter to emit light toward the test particles; and
a photodetector to determine a color of the dye coating from a measurement of voltage of the light reflecting from the test particles.

4. The apparatus as defined in claim 1, wherein the apparatus further comprises a blood extraction pad, the mixed blood and detection fluid are mixed in the blood extraction pad, and the particle flow controller removes the test particles from the blood extraction pad using an electromagnet.

5. The apparatus as defined in claim 1, wherein the allergen detector notifies a reaction preventer of the exposure to an allergen based on the color and the reaction preventer is to:
notify a user of the exposure to the allergen; or
treat the user by injecting the user with a drug via a treatment cannula.

6. The apparatus as defined in claim 1, wherein the allergen detector determines a severity of the exposure to the allergen based on a difference in the color of the magnetized test particles and a baseline measurement color of other magnetized test particles that was previously measured by the allergen detector.

7. The apparatus as defined in claim 1, further comprising a blood extractor to extract the blood from the user, wherein the extraction controller controls the blood extractor to extract the blood from the user at particular times, the blood extractor comprising
a blood extraction pad to receive the blood extracted from the user; and
an extraction cannula to extract the blood from the user subcutaneously.

8. The apparatus as defined in claim 1, further comprising a waste container, the waste container to receive the magnetized test particles after the allergen detector detects the exposure to the allergen based on a color of the test particles.

9. The apparatus as defined in claim 1, wherein the extractor controller controls a second extraction of blood after the exposure to the allergen is detected by the allergen detector to confirm that the exposure to the allergen has occurred.

10. A method comprising:
extracting, in response to instructions from a processor, blood from a user;
mixing the blood with a detection fluid to generate magnetic test particles, the detection fluid comprising dye coated magnetic particles;
measuring light reflected from the magnetic test particles using a photodetector; and
determining, via the processor, whether the user has been exposed to an allergen based on the measured light.

11. The method of claim 10, further comprising:
determining a color change between the measured light and previously measured light; and
determining a severity of an allergic reaction caused by the allergen based on the color change.

12. The method of claim 10, further comprising:
when the user has been exposed to the allergen, alerting the user of the exposure and providing treatment to the user by injecting a drug into the user subcutaneously.

13. A non-transitory computer readable storage medium comprising instructions that, when executed, cause a machine to at least:
instruct a blood extractor to extract blood from a user;
facilitate mixing the extracted blood with a detection fluid comprising dye coated magnetic particles;
control movement of the magnetic test particles to a photodetector; and
measure a voltage from light reflected from the magnetic test particles to detect the presence of an allergen in the extracted blood.

14. The non-transitory computer readable storage medium of claim 13, wherein the instructions, when executed, cause the machine to:
notify a user of an exposure to the allergen when the presence of the allergen is detected based on the voltage measured from the light.

15. The non-transitory computer readable storage medium of claim 13, wherein the instructions, when executed, cause the machine to:
treat the user by injecting a drug subcutaneously into the user when the presence of the allergen is detected based on the voltage measured from the light in order to limit an allergic reaction caused by the allergen, wherein the drug is injected by a treatment cannula inserted into the user.
